(19) United States
(12) Patent Application Publication
(10) Pub. No.: US 2010/0261991 A1
(43) Pub. Date: Oct. 14, 2010

(54) Apparatus for wire or wireless ECG machine with only two leads

(76) Inventors: Guangren Chen, Palm Harboor, FL (US); Drew Maisen Diaz, Colleyville, TX (US); Xiaohong Yang, Shanghai (CN)

Correspondence Address: Kavita Akula;Suite 225, 1303 W. Walnut Hill Lane, Irving, TX, 75038, US

(21) Appl. No.: 12/386,018

(22) Filed: Apr. 11, 2009

(51) Int. Cl. A61B 5/0408

(52) U.S. Cl. 600/386

(57) ABSTRACT

A disposable “Enhance Dynamic Flow Data” electrode, including: a flexible strips EDFD electrode and leads cable and voltage socket. Electrode with affixed program micro control chip, the chip having a positioned inside, the strips electrode have six electrode on one electrode with (V1,V2,V3,V4,V5,V6) attached of a receptor pad end connection with the skin for receiving electrical impulses and signal which is adapted for connection with an electro-cardiologram measuring on body chest apparatus. The specific enhance the dynamic flow signal configuration propriety for electrocardiography recording. An embodiment of the invention will be described in greater detail below with reference to the accompanying drawings which show an EDFD electrode and wire or wireless leads cable connect with device embodying the invention. Enhance bio-electrical signal's purity, thus enhances the test for more accuracy analysis finally.

### CROSS REFERENCE TO RELATED APPLICATION

### Field of the Invention

This invention is in the field of monitoring and diagnosing electrical activity within the human body.

### BACKGROUND

This application has to demonstrate the prior art, for example, in the heart, electrical signals coordinate the rhythmic pumping of the cardiac muscles and the bio-potential signals resulting from the heart's electrical activity are routinely recorded. This recordation of the well-coordinated electrical events that take place within the heart is called an electrocardiogram (ECG). There are many different types of ECG. However, rhythmic ECG signals with respect to various time and amplitude and frequency content are obtained from different regions of the heart. These regions around the organ include the conduct bundle and myocardium that generate various electrical activity bio-electric signals that travel throughout the human body surface. The purpose of this ‘Enhance Dynamic Flow Data’ (hereafter referred to as EDFD) electrode signal transmission is to distribute information from extra high impedance when a necessary function is being carried out. For example, body surface electrical phenomena currency exhibits only about 10% of the result of this electro-physiological activity in the presence of various bio-electric voltages. If using EDFD electrode a system may be able to obtain good ECG recordings. Clinically, the ECG technique is currently used to diagnose a number of physiological conditions. The status of heart muscles (e.g., potential abnormal ischemia) is not often detected and various life-threatening heart arrhythmias and ischemia are not routinely identified. The reason for non-identification for ischemia in pre-existing ECG systems is because the pre-existing systems are not sensitive enough to display this information. Specifically, the pre-existing systems lack the detection in “T” section of ultra low frequency relative to the time domain linear waveform; therefore, if detected, the information could be helpful for more pure signal and allow guidance on more specific testing modalities for the patient. Use of the ECG is especially widespread and the equipment is highly advanced for ischemia & infarct diagnosis. There is a sophisticated ischemic & infarct diagnostic ECG instrument, and ischemia monitoring devices for routine use for a variety of medical environments and even portable devices. In spite of the sophisticated ECG systems, the missing generated data still has a lot of promising information that has not been exploited to its maximum potentialities, and current system missing this generated data may not make a proper diagnosis of a heart condition. With little training, most medical staff cannot obtain a good deal of information from the ECG signals. Automated, computer analysis is a very accurate assistant to the Cardiologist in diagnosing various heart abnormalities. Because of the nature and convenience of obtaining and interpreting the ECG signals, all ECG systems are very different, but almost every patient in an operating room (OR), intensive care unit (ICU), or ER environment is routinely monitored with an ECG equipment, so the waveform settlement are very important. Therefore, it is highly desirable in the art to have a special electrode that provides a highly sensitive signal, micro-chip input that is indicative of the state of strip EDFD electrode. This input chip corresponds to a numeric value with respect to the heart activity to maximize heart's information activity, respectively.

Another shortcoming in the previous ECG systems are measuring the length of wires mainly related to the device and to patient, and then having these long wires tangled. The old ECG systems are used for routine patient monitoring, or screening normally obtained in the OR, ICU, or ER environment. When using the old ECG systems, the 12 leads ECG with 10 leads cables are inconvenient to use and to put on the patient. When monitoring or screening the patients, the lead cables must have good contact with the skin and must monitored in a special environment. The one strip with six electrodes are relatively simple to attach by an adhesive patch and easy to remove. (i.e., usually the upper-body clothing must be removed). Sometimes during use, the leads often became disconnected or out-of-order that requires re-set up. Such difficulties cause the following problems. A trained technical staff personnel is normally required to set up the leads. Often the 10 lead cables are winding and tangled at all times; therefore, resulting in wrongful connections of the leads and not being able to obtain the desired ECG data. There is an additional cost associated with using trained staff, and the current medical reimbursement policies tend to encourage minimal use of trained staff in the hospital. The attachment protocol of the lead cables take a considerable amount of time especially when multiple electrodes are required. The ordeal is difficult and tedious for the patient. Furthermore, if a patient were to be relocated in a different area in the hospital, all electrodes must be detached or the entire unit must relocate with the patient. Therefore, it is highly necessary in the art to use a Bluetooth module or a wireless configuration between patient that is connected to leads and device that monitors and displays information.

In hospital use of Magnetic Resonance Imaging (hereafter referred to as MRI) machines have become a routine method for obtaining information regarding a patient's anatomy and physiology. Currently, however, not many patients are monitored with an ECG instruments while they are in the MRI. Basically, the MRI and ECG equipment are not compatible. The operating MRI produces strong radio frequency (RF) fields and large static magnetic fields are always present. These fields create a strong magnetic flux that induces current flow in electrodes and to any attached electrode wires that form a loop. The result of the increase in the magnet flux has been reported or a resulted in instances of localized skin burns from the electrodes and looped wires residing in MRI machines. Therefore, causing current distribution that is diffused throughout the patient's body. Such situations can be fatal if the current induced is sufficiently large. The presence of equipment near the MRI machine can also interfere with the diagnostic quality of the MRI images themselves by causing distortions in the MRI output. Also, the radio-frequency (RF) fields of the MRI machine can corrupt the weak signals being recorded by ECG equipment and especially even weaker signals associated with 12 lead ECG instruments. For this reason, a special screen room is built around the MRI machine to prevent from affecting other equipment in the vicinity of the imaging device. Generally, all patients in OR, ER, ICU, CCU, have to remove all unnecessary equipment, and must keep it outside the screen room. To solve these and other associated problems with prior art measuring with EDFD electrodes, it is an object of the present invention to provide a better ECG waveform that avoids a macro electronic countermeasure in hospital situation. It is an object of the present invention to provide easy set up for using EDFD electrode.

### SUMMARY OF THE INVENTION

The present invention provides a solution comprises EDFD electrode with a support ‘Enhancement Dynamic Flow Data’ (EDFD) chip with amplifiers build in the middle of leads cable, so that the signal will be received from both the front buffer amplifier level and the high input resistance by the request from pre-amplifier allowing electrically insulated data even when the ECG signal has taken up a very weak signal from the body. Preferably, the arrangement is such that EDFD chip will also be a more effective and will enhance the bioelectrical signal between the electrode and the device, and remove disturbance from the electrode. The present invention creates a bioelectrical signal that will be clean without interference; such interferences can migrate under bioelectricity action and move up to the electrode causing false data. The EDFD chip programmed and support may include the EDFD electrode signal processing unit; if desired, the ECG signal may be more stable provided, separately or combined with the PCB-support, on the lead cables to the device when the EDFD electrode is being used. By using the present invention amplifiers, one can further enhance the EDFD electrode characteristics of the dynamic flow data. Typically, however, it is desirable that an electronic signal from EDFD electrode transform into an order to take advantage of amplifiers that provide bio-potential and basic signal. Specifically, the EDFD electrode produces an electronic signal in response and proportional to the present invention device, and preferably is used to control the flow of current in a circuit by means of an advance amplifier. One of the advantages of the present invention is to increase clear signal and decrease noise in proportion to the amount of current flowing in the detection circuit.

It is an object of the present invention to minimize patient preparation for EDFD electrode measurement. It is an object of the present invention to avoid lead cables looping and curling, or stirring. It is an object of the present invention to provide standard clinicians and hospital with an EDFD electrode that is easy to use without extensive training. It is an object of the present invention to provide two leads instead of ten line cables. It is an object of the present invention to minimize noise by EDFD electrode and two lead cables. It is an object of the present invention to provide a EDFD electrode instrument with low power consumption. It is an object of the present invention to provide an EDFD electrode that can be used at various sides with an output that interfaces with standard amplifiers, filters, hardware devices, and computer software.

### DESCRIPTION OF THE DRAWINGS

FIG. 1: is the systematic layout of the V1-V6, Bluetooth Moduel, and other embodiments of the present invention.

FIG. 2: is a schematic block diagram of the present invention and its lead cables connected wirelessly to the device.

FIG. 3: is a schematic block diagram of the present invention and its lead cables connected directly to the device.

FIG. 4: is an illustration of the view of the electrodes with cables labeling the specific components.

FIG. 5: is the connection of the male and female connection.

FIG. 6: is the layout of the heart and potential placement of the V1-V6 on the patient.

### DETAILED DESCRIPTION

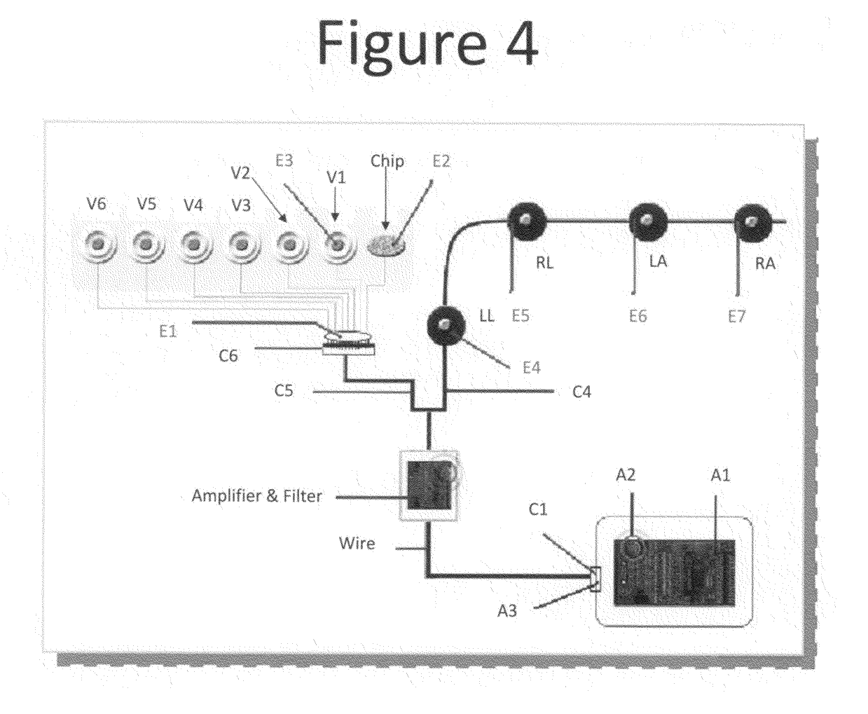

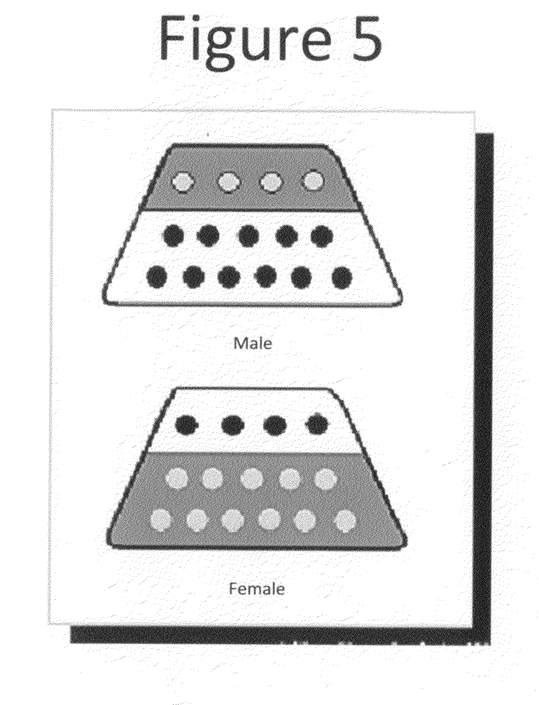

FIG. 4 is an illustration of the electrodes with cables connected to the PCB device. The items E3-E7 are items that are connected to the patient. The items are connected to the patient to allow the system to detect the micro-bioelectrical signals of the patient without collecting unnecessary interferences from the disturbance. The PCB device is used to connect and receive all the signals from the electrodes, and modulate the collected data, which is buffered to a TV monitor, LCD screen, or any display means, as disclosed on A1. Furthermore, the PCB device contains a Bluetooth modulator, item A2. The Bluetooth modulator is used to communicate wireless with nodes. The wireless communication allows easily mobility of the patient, allows a cost-effective method of reduction trained personnel, and prevents the wires from being tangled. The PCB device also includes a connector. The connector can be either male or female. The purpose of having the connector is to allow older systems to use PCB. There are two different connectors. The first connector is for chest leads, item A3, and second connector is for body leads, item C1. With respect to the electrodes, we have E3-E7 that connects to the patient, as was mention above. Specially, item E3 demonstrates V1, V2, V3, V4, V5, V6, which are connected to the patient's chest. The improvement of the design is for user's convenience; therefore, the present invention has six chest electrodes V1, V2, V3, V4, V5, V6 that are connected in the single lead line. The placement of the electrodes will be further described in FIG. 5. Next, item E1 and C6 are connectors; this connects the electrodes and leads module. The bioelectric signal is collected through the electrodes send to the leads module, which is then processed by the amplifier and is later filtered. After the signal processing is complete, the signal is transmitted through the standard of Bluetooth to the receiving transceiver Bluetooth in the PCB device, and converted to data to be displayed on a display means. Also, items E4-E7 are connected to the human body. LL is connected to left leg, item E4. LA is connected to the left arm, item E7. RA is connected to right arm, item E6. RL is connected to right leg, item E5. The connectors are branched from the body lead module, item C4. Finally, after the data is processed through the body lead module, the older system that are not equipped can send the data through via a wire connected to the PCB. A list of the items is stated below for a quick referral.
(A1) The part of PCB in device.
(A2) The Bluetooth module is on inside of PCB.
(A3) Connector (Between device and leads)
(C1) Connector (Between device and leads)
(C2) Leads cable side
(C3) LEADS Module
(C4) Body leads side
(C5) Chest leads side
(C6) Electrode connector at chest leads side
(E1) Electrode connector at chest leads side
(E2) Chip at chest leads side
(E3) Electrode with 6 connections V1-V6
(E4) LL connector (clip, button, snap, jack, clamps)
(E5) RL connector (clip, button, snap, jack, clamps)
(E6) RA connector (clip, button, snap, clamps)
(E7) LA connector (clip, button, snap, jack, clamps)

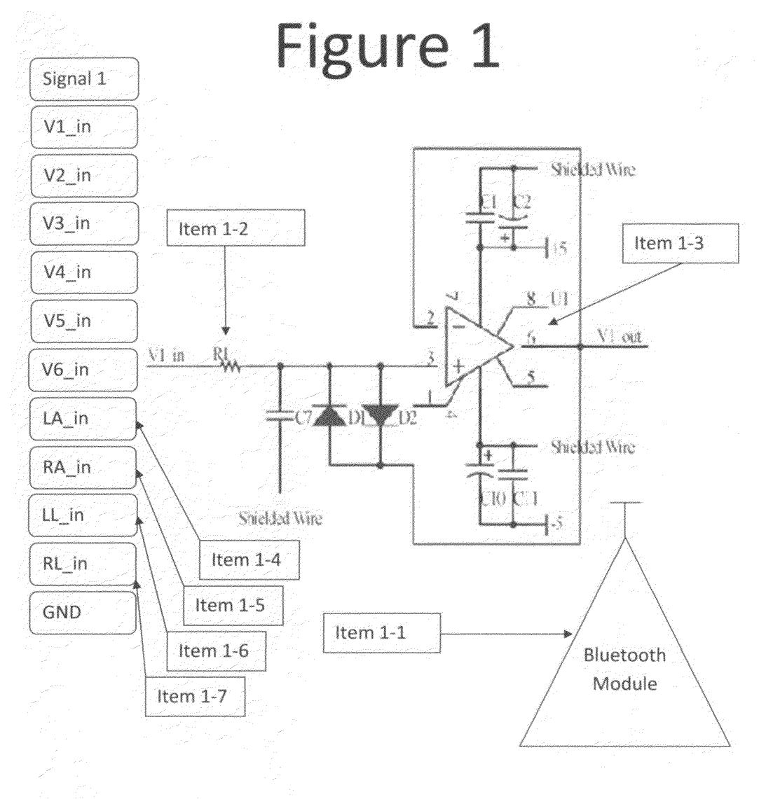

FIG. 1 is a block diagram of the present invention that is illustrating its major amplifier and filter components including a Bluetooth transmitter source, item 1-1, high impedance electro-signal modulator, item 1-2, and electronic amplifier circuitry, item 1-3. Item 1-2, the high impedance electronic-signal modulator is important because the heart electrical signal is very weak, the signal collected from the body surface is only 1-2 mV, moreover, taking the human body's heart as the electrical source. When only measuring such a small voltage, a system is susceptible to large amount of interference resistance. Therefore, the present invention provides a pre-amplifier to reduce high input resistance; otherwise, the measured signal will contain large amounts of error. The illustration is a schematic block diagram of the amplifier in the micro-PCB. The block diagram further demonstrates LA_in electrode signal input is on the left-hand of the patient, item 1-4, RA_in electrode signal input is on the right-hand of the patient, item 1-5, LL in electrode signal input is on the left-foot of the patient, item 1-6, RL in electrode signal input is on the right-foot of the electrode, item 1-7. V1_in—V6_in are the 6-electrode signal input data collection electrodes that are affixed on the chest of the patient. LA_out and V1_out are representatives of the output signal from the cushion amplifier circuit and the output buffer amplifier from LA_in and VI_in signal inputs, V10 is built into the chip of the chest electrodes, which controls and enlarges the main ECG signal with buffer output and then transmits through the Bluetooth standard. V11 is a Bluetooth module. VIa is a controller chip installed in V1_in—V6_in and primary controls the cushion enlargement heart electrical signals and the Bluetooth out, item VII. On various occasions by the hospital environment, the user can choose to use the wireless Bluetooth transmission or leads cable wire transmission.

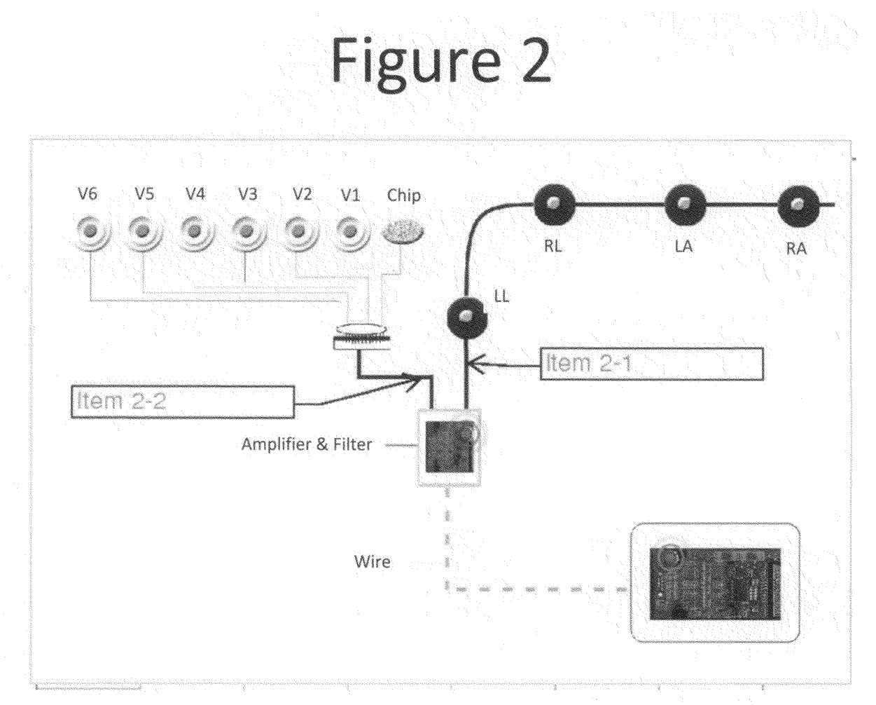

FIG. 2: is a schematic block diagram of the present invention and shows the lead cables connected wirelessly to the device. Further, the illustration shows the top view of a double cable line showing the placement of the 10 cables, one branch line cable is EDFD electrode, item 2-2 that is held in position, also illustrated in FIG. 4 item E3. The other branch line cable, item 2-1, is for body position, also illustrated in FIG. 4 items E4, E5, E6, and E7 which points to LA, RA, LL, RL (snap or pin) connect to any kind of electrode. The key feature of FIG. 2 is used to demonstrate that the filter and amplifier is connected wirelessly to the device. This is important since these lead cables can create a large amount of mobility for the patient.

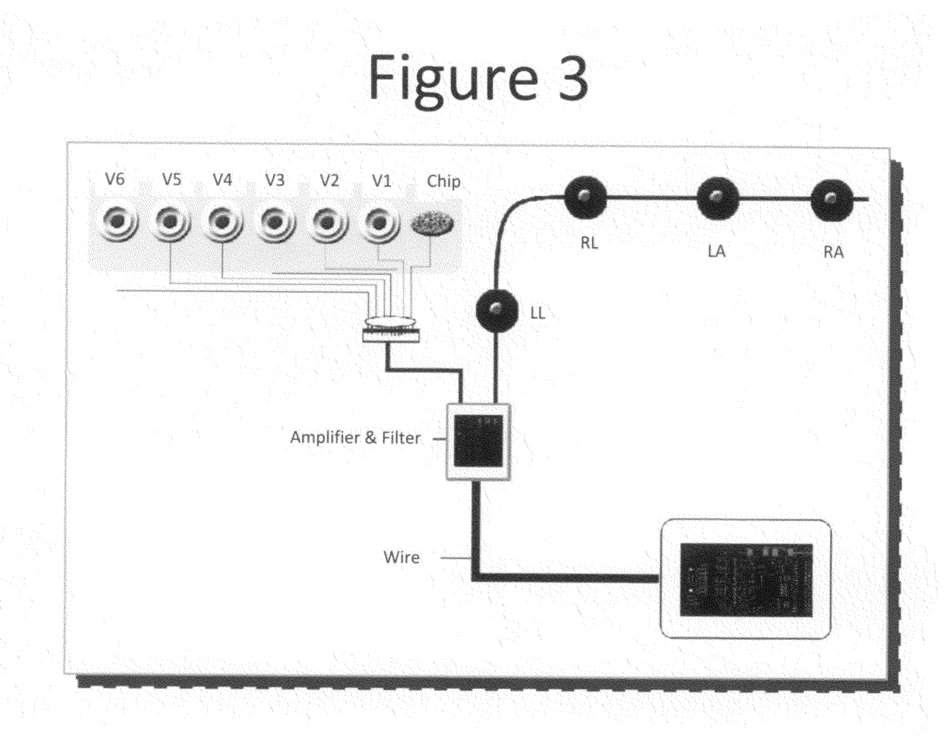

FIG. 3: is a schematic block diagram that shows the connection of the lead cables to the device. Attachment of EDFD electrodes to the device introduces new sets of problems for the medical staff wanting to use this technology. Specifically, the illustration shows how the system physically is connected to the device. The problems that arise are namely that 12 lead ECG often uses only 10 of the disposal electrodes, 6 pieces for chest and 4 pieces for the body; thus leaving two extra electrodes, which all look the same. This method is usually very problematic for medical staff because each cable needs to be identified and made sure they are connected to the patient properly. If a wrong cable is connected, this will cause problems for the device and give an inaccurate reading of the patient. Therefore, as compared to already existing system in the market, the present invention solves this problem by using only the EDFD electrodes that is connected to 6-pieces on one strip, and connecting only to one lead cable. The other four pieces of electrode only have one line lead cable that connects on Left Leg, Right Leg, Right Arm, and Left Arm to the patient's body; therefore, this application only uses one line lead cable shaped with four electrodes, instead of a replacement for four line lead cables. Thus, the two lead cables will avoid the improper connection of the chest electrodes to the arm and leg connections.

FIG. 5: is a side view of jacks, connecting the leads to the device and illustrating male and female jacks. The present inventions uses new connectors instated of common PIN connectors, because the PINS are easily damaged and present systems in the market are not convenient for insertion. Therefore, the present invention has been designed more convenience and simple connector. Furthermore, the benefit of the present invention allows for fast set-up time, and as well as reduces the cost of damage PINS.

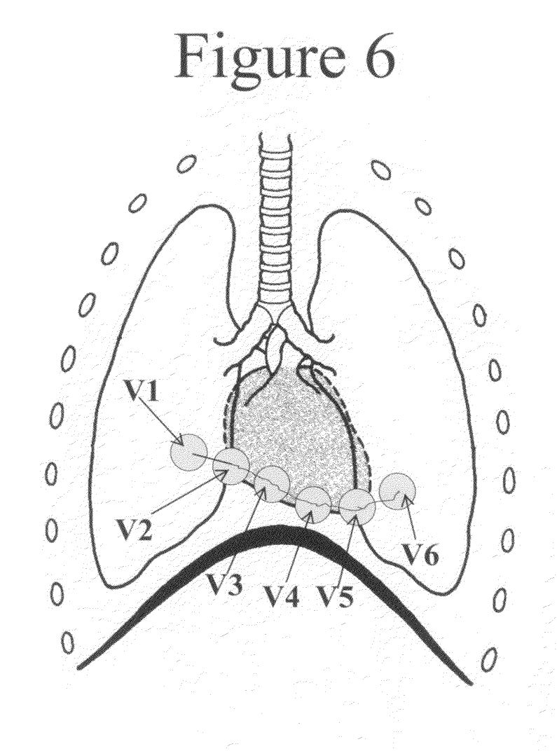

Furthermore, an illustration of FIG. 6 is the amplifier part, visible light, item 22, from light source, item 20, passes through the electro-optic modulator, item 30, without changing the absence of a bio-potential. The bio-potential is a voltage or the current strength caused by the change in the ions. Also, the bio-potential is used to exchange information between the cells or compression of the organ that is truncated into a signal. However, when electro-optic modulator, item 30, is placed in the presence of a bio-potential, for example positioned near the heart of a patient, the changes in bio-potential produced by the heart as it beats causes modulator, item 30, to change illumination property of the light. For example, if the intensity of the light recorded is the illumination property being changed under the influence of the bio-potential, light, item 32, emerging from modulator, item 30, is observed by the naked eye to become brighter or darker with each beat of the heart. Thus, in this basic form, one can determine the heart rate of the patient by counting the number of changes in the illumination property per unit of time. Since, many bio-potentials have multiple inputs, for example the ECG waveforms on the surface of the heart as recorded in an ECG, the present invention provides for the use of multiple electrodes on one strip, which can contain six in one. At this end of the process, the program micro-controller chip then uses the amplifier to get additional informatics provided by cushion amplifying circuit. Specifically, The micro-controller chip function is used to enhance certain signals. The heart's electrical signal obtained from the human body surface contains 10% of the actual heart's electrical signal, and less than 1% of the actual myocardium signal is obtained from the electrocardiogram analysis; therefore, the present invention enhances certain signals that are important. The interference to the heart's electrical signal includes the baseline drifting and the power frequency interference, these may be reduced through the buffer amplifying level. Thus, the function of the micro-controller chip in the present invention represents mainly in the following three aspects: 1) to coordinate and discriminate the chest electrode line of 6 group signal inputs; 2) to control the nominal operation stably of the buffer amplifier; and 3) to control the signal launch of the Bluetooth module. The present invention designs the middle part of the lead cables. The lead cables group both the heart electrical signals of cushion amplifying circuit and the Bluetooth transmission circuit. The signal is mixed with some interference when the heart signal is extracted from the human body surface. The present invention allows the heart electrical signal to be examined undistorted by the system through the body surface sensor by removing the strong noise background. The enlargement part divides into various levels, in the middle pm of the leads mainly involve the front buffer amplifier level. The front buffer amplifier level goes before the pre-stage process.

In conclusion, the overall EDFD electrodes and two separate lead lines to the patient are an improvement over the standard techniques used to this day. The improvement features of the present invention include the method of a more consistence signal where the high-impedance contact requires no special or skin abrasion preparation that directly decreases unwanted signal from by using EDFD electrode. Next, the low cost of the present invention compared with presently commercially available products take too much time and money to prepare. Another improvement is that the present invention has two lead lines instead of the standard 10 lines cables. This is an improvement since the lead lines are easily decipherable and will result in less misplacement of the lines that causes direct result of accurate data. Another improvement of the present invention is the capability to measure unsteadiness bio-waveform available at various frequencies, and offering an easy set-up for the patient. The set-up is an improvement because it consolidates all the 10 multiple lines into 2 line lead cables, specially 4 on one the body lead cable and 6 on the chest lead cable. Another improvement of the present invention is retention compatibility strength from emanating organ compared to the standard commercially available filtering, amplification, and hardware. The present invention allows the capture of more data fluctuations by software programs on controller chip. The controller chip takes the data, determines the modulation noise compared to the actual modulation data from the chest lead cable and displays that data in the form of illumination property on a screen.

In describing the invention, which is illustrated in the drawings, specific terminology is presorted to the chip for clarity. However, it was not intended that the invention be limited to the specific terms so selected and it is to be understood that each specific term includes all technical equivalents that operate in a similar manner to accomplish a similar purpose.

Although preferred EDFD electrode and wire or wireless leads cable of the invention have been herein described, it is understood that various changes and modifications in the illustrated and described structure can be affected without departure from the basic principles that underlie the invention. Changes and modifications of this type are, therefore, deemed to be circumscribed by the spirit and scope of the invention, except as the same may be necessarily modified by the appended claims or reasonable equivalents thereof.

1. A disposable cable apparatus used to communicate to a PCG device comprising:
a flexible conductive strip;
a chest lead cable;
a body lead cable;
a Bluetooth modulator that communicates to said PCG device;
a micro-controller chip, wherein said micro-controller chip can be located in a fixed position and receiving and transmitting clear electrical impulses, which is adapted for connection with an electromyo-cardiogram measuring apparatus; and
a plurality of enhance dynamic flow data electrodes affixed thereon in a specific configuration appropriate for electromyo-cardiogram signal retrieval.

2. A disposable cable apparatus according to claim 1, wherein said chest lead cable comprises said plurality of enhance dynamic flow data electrodes.

3. A disposable cable apparatus according to claim 1, wherein said body lead cable comprises a plurality of body electrodes, wherein said plurality of body electrodes are connected all over the patient's body.

4. A disposable cable apparatus according to claim 1, wherein said plurality of enhance dynamic flow data electrode are built in said flexible conductive strip electrode.

5. A disposable cable apparatus according to claim 1, wherein said chest lead cable and said body lead cable are connected to said Bluetooth modulator.

6. A disposable cable apparatus according to claim 1 comprising said micro-controller chip having a predetermine size and name and design.

7. A disposable cable apparatus according to claim 1 comprising, wherein said flexible conductive strips contains said plurality of enhance dynamic flow data electrodes that are pre-program with said micro-controller chip build in circumference of said plurality of enhance dynamic flow data electrodes.

8. A disposable cable apparatus according to claim 1 comprising, wherein said plurality of enhance dynamic flow data electrodes, said micro-controller chip, and said plurality of body electrodes have a company logo and said company software design.

9. A disposable cable apparatus according to claim 1 comprising, wherein said flexible conductive strip is at least from cellulosic materials, polyesters, polyolefins, polyvinyl chloride, or nylon.

10. A disposable cable apparatus according to claim 9, wherein said cellulosic materials contains one or more of cotton, paper, or polyester is polyethylene.

11. A disposable cable apparatus according to claim 9, wherein said plurality of enhance dynamic flow data electrodes is composed of one or more of a metal, a polymer, a graphite, or a carbon fibers.

12. A disposable cable apparatus according to claim 11, wherein said metal contains one or more of gold, copper, silver, tin, aluminum and alloys, or any combination thereof.

13. A disposable cable apparatus according to claim 9, wherein said enhance dynamic flow data electrode are made of metal foil or a metal paste.

14. A disposable cable apparatus according to claim 9, wherein a metal paste is printed or silk screened onto a conductive sheet.

15. A disposable cable apparatus according to claim 9, wherein said enhance dynamic flow data electrode contains a electoral chip deposited on said conductive sheet.

16. A disposable cable apparatus according to claim 11, wherein said polymer is selected from the group consisting of polyesters, copolymer of ethylene-vinyl acetate, homopolymers and copolymers of polyvinylchloride and ABS resin.

17. A disposable cable apparatus according to claim 1, wherein said Bluetooth modulator is connected wirelessly with a wireless amplifier and wireless filter.

17. A disposable cable apparatus used to communicate to a PCG device comprising:
a flexible conductive strip, wherein said flexible conductive strip is used to attach to the chest of a patient;
a chest lead cable, wherein said chest lead cable is connected in a predetermine sequence on said patient;
a body lead cable, wherein said body lead cable is connect to a right-arm, a left-arm, a right-leg, and a left-leg of said patient;
a Bluetooth modulator that communicates to said PCG device, wherein the said Bluetooth modulator is connected with wireless an amplifier and a wireless filter;
a micro-controller chip, wherein said micro-controller chip can be located in a fixed position and receiving and transmitting clear electrical impulses, which is adapted for connection with an electromyo-cardiogram measuring apparatus; and
a plurality of enhance dynamic flow data electrodes affixed thereon in a specific configuration appropriate for electromyo-cardiogram signal retrieval, wherein said plurality of enhance dynamic flow data electrode are built in said predetermined sequence on said flexible conductive strip.

18. A method of fitting a patient with a connector leads cable comprising attaching a strip of a plurality of enhance dynamic flow data electrodes;
attaching a body lead cable comprising a plurality of body electrodes in a predetermine position on the human body;
receiving and transmitting electrical impulses through said plurality of enhance dynamic flow data electrodes ends V1, V2, V3, V4, V5 and V6 positioned at predetermined fixed locations;
amplifying electrical signal from said plurality of enhance dynamic flow data electrodes;
connecting a device to said body lead cable and said chest lead cable;
measuring between said device and said plurality of enhance dynamic flow data on the chest of the patient;
calculating a illumination property depending on the measurement between said device and said plurality of enhance dynamic flow data and said body lead cable; and
displaying said illumination property respect to time on the display apparatus.
